(19) United States
(12) Patent Application Publication
(10) Pub. No.: US 2026/0024219 A1
(43) Pub. Date: Jan. 22, 2026

(54) APPARATUS FOR DETECTING CARBIDE PARTICLE IN STEEL MATERIAL, METHOD FOR DETECTING CARBIDE PARTICLE IN STEEL MATERIAL, AND PROGRAM FOR DETECTING CARBIDE PARTICLE IN STEEL MATERIAL

(71) Applicants: NHK SPRING CO., LTD., Yokohama-shi (JP); NATIONAL UNIVERSITY CORPORATION TOKAI NATIONAL HIGHER EDUCATION AND RESEARCH SYSTEM, Nagoya (JP)

(72) Inventors: Shintaro KUMAI, Yokohama-shi (JP); Keita TAKAHASHI, Yokohama-shi (JP); Kosuke ONUMA, Yokohama-shi (JP); Yoshitaka ADACHI, Nagoya-shi (JP)

(21) Appl. No.: 19/343,598

(22) Filed: Sep. 29, 2025

Related U.S. Application Data
(63) Continuation of application No. PCT/JP2024/012606, filed on Mar. 28, 2024.

(30) Foreign Application Priority Data
Mar. 31, 2023 (JP) 2023-057525

(51) Int. Cl. G06T 7/50; G01N 23/2251; G01N 33/2028; G06T 7/12; G06T 7/136; G06T 7/155; G06T 7/60

(52) U.S. Cl. CPC G06T 7/50; G01N 23/2251; G01N 33/2028; G06T 7/12; G06T 7/136; G06T 7/155; G06T 7/60; G06T 2207/10061; G06T 2207/20036; G06T 2207/30136

(57) ABSTRACT

There is provided an apparatus for detecting a carbide particle capable of properly identifying carbide particles in a steel material, a method for detecting the carbide particle capable of properly identifying the carbide particles in the steel material, or a program for detecting a carbide particle capable of properly identifying the carbide particles in the steel material. An apparatus for detecting a carbide particle according to an embodiment of the present invention includes an extraction unit for binarizing image data of a microscope image of a steel material to extract shapes of carbides, and a separation unit for separating particles of the carbides from the binarized image data based on watersheds or the shapes of the carbides.

### CROSS-REFERENCE TO RELATED APPLICATIONS

This application is a Continuation of International Patent Application No. PCT/JP2024/012606, filed on Mar. 28, 2024, which claims the benefit of priority to Japanese Patent Application No. 2023-057525, filed on Mar. 31, 2023, the entire contents of which are incorporated herein by reference.

### FIELD

The present invention relates to an apparatus for detecting a carbide particle of a steel material. Alternatively, the present invention relates to a method for detecting a carbide particle of a steel material. Alternatively, the present invention relates to a program for detecting a carbide particle of a steel material.

### BACKGROUND

As one of the indexes for characterization of a steel material, carbide morphology is used. Generally, the carbide morphology in the steel material exposes a structure of the steel material by etching, and by observations using a scanning electron microscope (SEM), the carbide morphology is evaluated by extracting features such as the size and number of carbides. In order to extract the features of the carbide, although SEM images are binarized based on luminance to extract carbide particles, binarization makes it impossible to properly extract individual particles, and a structure in which multiple particles are bonded together as if they were a single particle is detected, making it difficult to properly evaluate the size and number of carbides.

As an approach for properly evaluating a structure of a metallic material by image processing, for example, Japanese Laid-Open Patent Publication No. 2021-149222 describes an image processing method for estimating continuous grain boundaries from an image in which the grain boundaries are unclear and disconnected, wherein the image processing method includes a first image processing in which a set of data strings of luminance values on one line in at least one direction along the image plane is automatically determined for an image obtained by photographing the surface of a sample using a reaction-diffusion equation whose solution state changes over time, and binarization is performed with a directional element based on a threshold parameter in the set, and a second image processing which generates a first tessellation image showing ridge lines, apexes, or contour lines of a geography when boundaries between grains are assumed to be channels by performing Euclidean distance mapping and watershed processing on the image obtained by the first image processing, and generating a second tessellation image showing boundaries between grains as the channels estimated from the ridge lines apexes, or contour lines of the geography by performing the Euclidean distance mapping and the watershed processing on the first tessellation image again.

### SUMMARY

However, similar to the grains evaluated in Japanese Laid-Open Patent Publication No. 2021-149222, since carbides in the steel material are the linear structure contained in the particles of the steel material unlike a structure having a certain degree of a cross-sectional area, an appropriate detection method using image processing has not been studied so far.

An object of an embodiment of the present invention is to provide an apparatus for detecting a carbide particle that can properly identify carbide particles in a steel material. Alternatively, an object of an embodiment of the present invention is to provide a method for detecting a carbide particle that can properly identify the carbide particles in the steel material. Alternatively, an object of an embodiment of the present invention is to provide a program for detecting a carbide particle that can properly identify carbide particles in the steel material.

An apparatus for detecting a carbide particle according to an embodiment of the prevent invention includes an extraction unit for binarizing image data of a microscope image of a steel material to extract shapes of carbides, and a separation unit for separating particles of the carbides from the binarized image data based on watersheds or the shapes of the carbides.

The separation unit may set a length threshold for separating the particles of the carbide based on a length range of the particles of the carbide determined from a known particle size distribution of the particles of the carbides.

The separation unit may separate a first portion and a second portion as different particles of the carbides when the separation unit identifies that the first portion of the shapes of the carbides and the second portion intersecting the first portion exist.

The extraction unit may include a characteristic value distribution extraction unit, the characteristic value distribution extraction unit may extract a distribution of characteristic values of the carbides from the image data of the microscope image of the steel material, the separation unit may execute a logic synthesis of the binarized image data and the distribution of characteristic values of the carbides to generate a logic synthesized image, and the separation unit may separate the particles of the carbides by using the logic synthesized image based on the watersheds.

A method for detecting a carbide particle according to an embodiment of the prevent invention uses an apparatus for detecting the carbide particle including an extraction unit and a separation unit, wherein the extraction unit binarizes image data of a microscope image of a steel material to extract shapes of carbides, and the separation unit separates particles of the carbides from the binarized image data based on watersheds or the shapes of the carbides.

The separation unit may set a length threshold for separating particles of the carbide based on a length range of the particles of the carbide determined from a known particle size distribution of the particles of the carbides.

The separation unit may separate a first portion and a second portion as different particles of the carbides when the separation unit identifies that the first portion of the shapes of the carbides and the second portion intersecting the first portion exist.

The extraction unit may include a characteristic value distribution extraction unit, the characteristic value distribution extraction unit may extract a distribution of characteristic values of the carbides from the image data of the microscope image of the steel material, the separation unit may execute a logic synthesis of the binarized image data and the distribution of characteristic values of the carbides to generate a logic synthesized image, and the separation unit may separate particles of the carbides by using the logic synthesized image based on the watersheds.

A storage media storing a program for detecting a carbide particle according to an embodiment of the prevent invention causes a computer to binarize image data of a microscope image of a steel material to extract carbides, and causes the computer to separate particles of the carbides from the binarized image data based on watersheds or the shapes of the carbides.

The program may cause the computer to set a length threshold for separating particles of the carbide based on a length range of the particles of the carbide determined from a known particle size distribution of the particles of the carbides.

The program may cause the computer to separate a first portion and a second portion as different particles of the carbides when the computer identifies that the first portion of the shapes of the carbides and the second portion intersecting the first portion exist.

The program may cause the computer to extract a distribution of characteristic values of the carbides from the image data of the microscope image of the steel material, the program may cause the computer to execute a logic synthesis of the binarized image data and the distribution of characteristic values of the carbides to generate a logic synthesized image, and the program may cause the computer to separate particles of the carbides by using the logic synthesized image based on the watersheds.

### BRIEF DESCRIPTION OF DRAWINGS

FIG. 1 is a block diagram showing an apparatus 100 for detecting carbide particles according to an embodiment of the present invention.

FIG. 2A is a SEM image of a surface of a steel structure exposed by etching.

FIG. 2B is a binarized image of a SEM image.

FIG. 3 is a flow diagram of a method for detecting carbide particles according to an embodiment of the present invention.

FIG. 4 is a flow diagram of a method for detecting carbide particles according to an embodiment of the present invention.

FIG. 5 is a flow diagram of a method for detecting carbide particles according to an embodiment of the present invention.

FIG. 6 is a flow diagram of a method for detecting carbide particles according to an embodiment of the present invention.

FIG. 7A is a SEM image of a surface of an exposed steel structure by etching of an example of the present invention.

FIG. 7B is a binarized image of a SEM image of an example of the present invention.

FIG. 7C is a diagram showing identified carbide particles according to an example of the present invention.

FIG. 8A is a diagram obtained by binarizing image data of a SEM image of a steel material of an example of the present invention.

FIG. 8B is an enlarged view of a part enclosed by a square in FIG. 8A.

FIG. 8C shows five particles of carbides identified in accordance with an example of the present invention.

FIG. 9 is a block diagram showing an apparatus 100A for detecting carbide particles according to an embodiment of the present invention.

FIG. 10 is a flow diagram of a method for detecting carbide particles according to an embodiment of the present invention.

FIG. 11 is a flow diagram of a method for detecting carbide particles according to an embodiment of the present invention.

FIG. 12 is a flow diagram of a method for detecting carbide particles according to an embodiment of the present invention.

FIG. 13 is a flow diagram of a method for detecting carbide particles according to an embodiment of the present invention.

FIG. 14 is a flow diagram of a method for detecting carbide particles according to an embodiment of the present invention.

FIG. 15 is a flow diagram of a method for detecting carbide particles according to an embodiment of the present invention.

FIG. 16 is a SEM image of a surface of an exposed steel structure by etching of an example of the present invention.

FIG. 17A is binarized image data of an example of the present invention.

FIG. 17B is a diagram showing an object group of identified carbides according to an example of the present invention.

FIG. 18A is a diagram showing an object of one carbide in which image data of a SEM image of a steel material is binarized according to an example of the present invention.

FIG. 18B is a diagram in which the object of the one carbide of FIG. 18A is distance transformed according to an example of the present invention.

FIG. 18C is a SEM image of a steel material of one carbide extracted from FIG. 16 corresponding to the one carbide of FIG. 18A according to an example of the present invention.

FIG. 18D is a logic synthesized image generated by a logical product of distance transformed image data and a distribution of characteristic values of carbides according to an example of the present invention.

FIG. 19A is a view of extracting distinct foreground regions for normalized logic synthesized images of an example of the present invention.

FIG. 19B is a diagram in which a watershed algorithm of an example of the present invention is applied to divide carbide structures and distinguish them as particles of different carbides, respectively.

FIG. 19C shows an application of a watershed algorithm using only distance transformed data.

### DESCRIPTION OF EMBODIMENTS

Hereinafter, embodiments of the present invention will be described with reference to the drawings. The embodiments shown below are an example of the embodiments of the present invention, and the present invention is not limited to these embodiments.

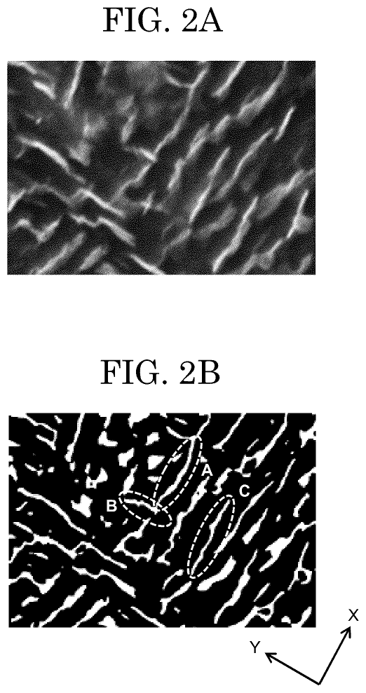

FIG. 2A is a SEM image of a surface of a steel structure exposed by etching. In FIG. 2A, the structure represented by white (high luminance) shows carbides contained in a steel material. In order to extract features such as the size and the number of carbides, an image in which the SEM image is binarized is shown in FIG. 2B. In FIG. 2B, it is observed that carbides having a linear structure toward two intersecting directions (an X direction and a Y direction) are arranged. Carbide particles contained in the steel material are characterized by being arranged toward the X direction or Y direction, and the structures of the carbides indicated by A and B in FIG. 2B are different carbide particles, respectively. However, in the image where the SEM image is binarized, two particles are observed as one combined structure, and it is impossible to distinguish that they are differing particles. Further, the structure of the carbide shown by C is observed as one structure in which a plurality of carbide particles arranged toward the X direction are combined. In order to properly evaluate the size and number of carbides, it is necessary to properly identify the carbide particles in the steel material.

### First Embodiment: Apparatus for Detecting Carbide Particle

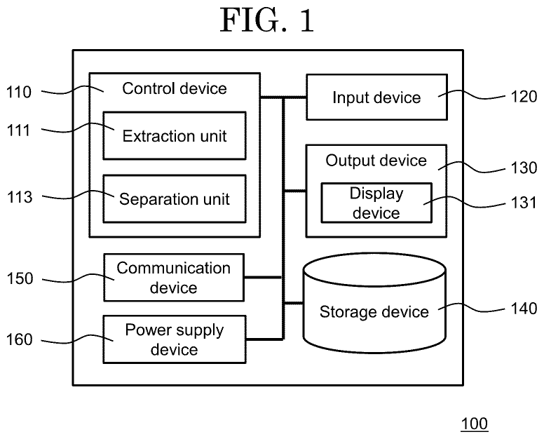

FIG. 1 is a block diagram showing an apparatus 100 for detecting a carbide particle according to an embodiment of the present invention. The apparatus 100 for detecting the carbide particle includes, for example, a control device 110, an input device 120, an output device 130, a storage device 140, a communication device 150 and a power supply device 160. Further, in an embodiment, the apparatus 100 for detecting the carbide particle, for example, further includes an extraction unit 111 and a separation unit 113.

The control device 110 is comprised of a known central processing unit (CPU), an operating system (OS) and control programs or modules for controlling the apparatus 100 for detecting the carbide particle. Alternatively, the control device 110 may be provided as one program that includes the OS and the control program or module. The control program or module that constitute the control device 110 is stored in the storage device 140 and executed by the CPU.

In FIG. 1, as an embodiment, although a configuration in which the control device 110 comprises the extraction unit 111 and the separation unit 113 is shown, the extraction unit 111 and the separation unit 113 may not be included in the control device 110 and may be arranged together with the control device 110.

The extraction unit 111 is composed of a program or module for binarizing the image of the SEM image of the steel material and extracting the shape of the carbides. The program or module constituting the extraction unit 111 is stored in the storage device 140 and executed by the CPU. In an embodiment, the extraction unit 111 binarizes a luminance value in the image data of the SEM image of the steel material, with respect to a predetermined value (hereinafter, also referred to as a first luminance value). More specifically, a luminance value of a portion having the luminance of a first luminance value or more (or larger than the first luminance value) in the image data of the SEM image is converted to 1, and a luminance value of a portion having the luminance less than the first luminance value (or of the first luminance value or less) is converted to 0. By such a process, the extraction unit 111 can identify a portion having a luminance value 1 as a structure of carbides. In addition, the first luminance value may be a luminance value set in advance from observation of the SEM image of the steel material, and may be a luminance value set by a user for the actual SEM image of the steel material to be processed by the extraction unit 111 and confirmed by the user on a display device 131. In an embodiment, an average value of luminance of a pixel of interest and luminance of surrounding pixels may be set as the first luminance value (adaptive binarization).

The separation unit 113 is configured with a program or module for separating the particles of the carbide from the image data binarized by the extraction unit 111. The program or module that configures the separation unit 113 is stored in the storage device 140 and is executed by the CPU. In an embodiment, the separation unit 113 separates the structure of the carbide identified by the extraction unit 111 into the particles of the carbide. In an embodiment, the separation unit 113 separates the particles of the carbide from the binarized image data based on a watershed or a shape of the carbide.

In the case of separating the particles of the carbide from the binarized image data based on the watershed, the separation unit 113 generates a gradation (distribution of luminance) toward the inside of the linear carbides in accordance with a distance from a background having a luminance value 0. For the generated gradation, a luminance value of a portion below a threshold of a predetermined luminance is converted to 0. Thus, the separation unit 113 extracts only the gradation portion of the gradation that is equal to or more than the threshold of the predetermined luminance from the gradation. The separation unit 113 sets the portion having the highest luminance among the extracted gradation portion as the center of the carbide. For example, in FIG. 2B, since a center is set in each of a portion A and a portion B of the carbide, the separation unit 113 can identify the center of the carbide portion A and the center of the carbide portion B as the centers of different particles of the carbide. Furthermore, the separation unit 113 can distinguish a carbide structure including the center of the portion A from a carbide structure including the center of the portion B as the different particles of the carbide.

In the case of separating the particles of the carbide from the binarized image data based on the shape of the carbide, the separation unit 113 detects a contour of the binarized structure of the carbide. Since the structure of the carbide has a linear structure, the structure of the carbide has a length direction and a width direction. The separation unit 113 detects a position where the width direction is shortened as a boundary of the particles of the two carbides (separation point) in the structure of the binarized carbides, and can identify the two particles of the carbides.

Further, there is a particle size distribution in the particles of the carbide, and the particles of the carbide have a range of length from this particle size distribution. In an embodiment, the separation unit 113 may set a length threshold for separating the particles of the carbide based on a length range of the particles of the carbide determined from the known particle size distribution of the particles of the carbide.

Further, as described for FIG. 2B, in the case where there are two portions having a length direction in each of the two intersecting directions, these portions are particles of carbides different from each other. In an embodiment, in the case where the separation unit 113 identifies the presence of the first portion (for example, the portion A in FIG. 2B) of the shape of the carbide and the second portion (for example, the portion B in FIG. 2B) intersecting the first portion, the separation unit 113 can separate the first portion and the second portion as the different particles of the carbide.

The input device 120 is a device for operating the apparatus 100 for detecting the particles of the carbide, and may be any known input device such as a keyboard, mouse, and a touch panel disposed on a display device (for example, a liquid crystal display or an organic EL display). In an embodiment, the input device 120 may include a scanning-electron microscope for acquiring the SEM images as described above. Alternatively, the input device 120 may include a drive or reader, such as a CD drive, a DVD drive, or a memory card reader, to which the media containing the image data of the SEM image can be connected.

The output device 130 includes the display device 131 for displaying various images generated by the apparatus 100 for detecting the carbide particle. The display device 131 can display, for example, a SEM image, mask data, a cross-sectional region of the crystal grains constituting the steel material, binarized image data, and the like. As the display device 131, for example, although a liquid crystal display, an organic EL display, or the like can be used, the display device 131 is not limited thereto. The output device 130 may also include a printer that prints an image displayed by the display device 131.

The storage device 140 is a device for storing an operating system (OS) and a control program or module that constitutes the control device 110, a program or module that constitutes the extraction unit 111, and a program or module that constitutes the separation unit 113. The storage device 140 is composed of, for example, a known main storage device, such as a random-access memory (RAM), a known auxiliary storage device, such as a read only memory (ROM), a hard disk or a solid state drive (SSD), and a memory card. In addition, the auxiliary storage device may be arranged outside the apparatus 100 for detecting the carbide particle, and may be arranged in a communicable server or a network drive which can communicate through the communication device 150.

The communication device 150 is a known wired or wireless communication device that is controllable by the control device 110. The communication device 150 may be connected to a communication network such as a local area network (LAN), a wide area network (WAN), and the Internet. The communication device 150 may be, for example, a communication device conforming to a wireless communication standard such as Wi-Fi (registered trademark) (a communication means using IEEE 802.11 standard) or Bluetooth (registered trademark). The communication device 150 may perform data communication with a server or a network drive arranged outside the apparatus 100 for detecting the carbide particle. In an embodiment, the communication device 150 may include a serial bus, such as a universal serial bus (USB), PCI Express and serial ATA (SATA), and a parallel bus, such as a small computer system interface (SCSI) and peripheral component interconnect (PCI).

The power supply device 160 is a device for supplying power from the outside to each device of the apparatus 100 for detecting the carbide particle, and it is not particularly limited.

### [Method for Detecting Carbide Particle]

A method for detecting a carbide particle using the apparatus 100 for detecting the carbide particle according to the present invention described above will be described.

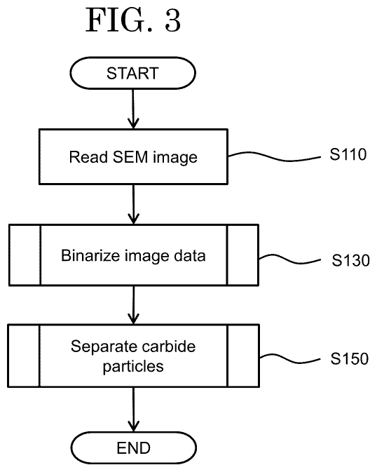

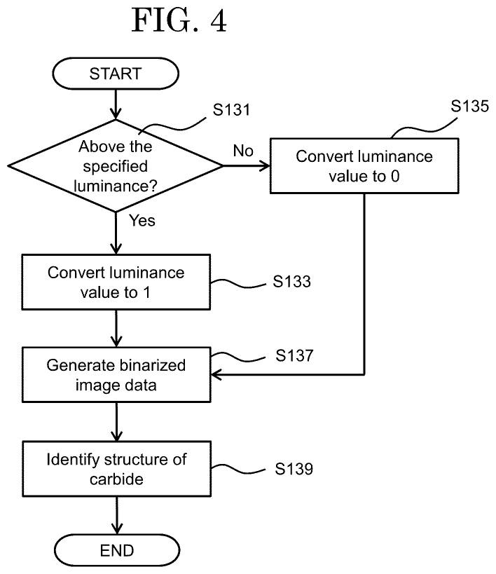

FIG. 3 to FIG. 6 are flow diagrams of a method for detecting a carbide particle according to an embodiment. The extraction unit 111 reads the SEM image of the steel material (S110). The SEM image of the steel material may be read through the input device 120, and the SEM image stored in the storage device 140 may be read by the extraction unit 111. Further, the SEM image stored in the server or the network drive may be read by the extraction unit 111 through a network connected to the communication device 150.

The extraction unit 111 binarizes the read image data of the SEM image of the steel material, and extracts the shapes of the carbides (S130). Specifically, the extraction unit 111 binarizes the image data of the SEM image of the steel material based on the first luminance value for each pixel constituting the image data of the SEM image of the steel material (S131). More specifically, the extraction unit 111 binarizes the luminance value in the image data of the SEM image of the steel material based on a predetermined value (hereinafter also referred to as the first luminance value). More specifically, the extraction unit 111 converts the luminance value of a portion having a luminance equal to or greater than the first luminance value (or greater than the first luminance value) in the image data of the SEM image to 1 (S133), and converts the luminance value of a portion having a luminance less than the first luminance value (or equal to or less than the first luminance value) to 0 (S135). The parts thus set are synthesized, and the extraction unit 111 generates image data in which the image data of the SEM image of the steel material is binarized (S137). Consequently, the extraction unit 111 can identify the portion having the luminance value 1 as the structure of the carbide (S139). In addition, the first luminance value may be a luminance value set in advance from the observation of the SEM image of the steel material, and may be a brightness value set by the user for the actual SEM image of the steel material to be processed by the extraction unit 111 and confirmed by the user on the display device 131. In an embodiment, the average value of the luminance of the pixel of interest and the luminance of the surrounding pixels may be set as the first luminance value (adaptive binarization).

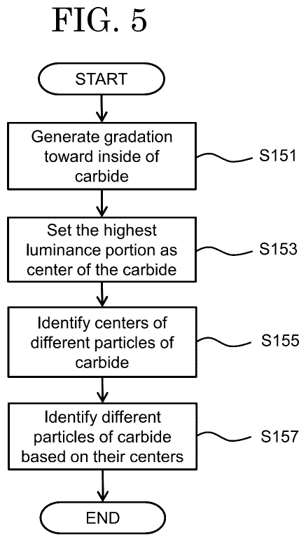

The separation unit 113 separates the particles of carbides from the image data binarized by the extraction unit 111 (S150). Referring to FIG. 5, a method for separating the particles of a carbide from binarized image data based on a watershed will be described. The separation unit 113 generates a gradation toward the inside of the linear carbides (distribution of luminance) in accordance with a distance from a background having the luminance value 0 (S151). The separation unit 113 sets the highest luminance portion of the generated gradation as the center of the carbide (S153). The separation unit 113 identifies a first center of the first portion of the carbide and a second center of the second portion of the carbide respectively as the center of the particles of differing carbides (S155). Furthermore, the separation unit 113 identifies a structure of the carbide including the first center and a structure of the carbide including the second center as particles of differing carbides (S157).

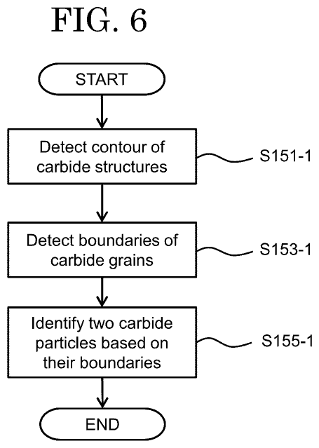

Referring to FIG. 6, a method for separating the particles of the carbide from the binarized image data based on the shape of the carbides will be explained. The separation unit 113 detects a contour of the structure of the binarized carbides (S151-1). The separation unit 113 detects a position where the width direction is shortened as the boundary of the two grains of the carbides (separation point) in the structure of the binarized carbide (S153-1). The separation unit 113 identifies the two grains of the carbides based on the boundary (S155-1).

### Modified Example of Method for Detecting Carbide Particle

There is a particle size distribution in the particles of the carbide, and the particles of the carbide have a range of length from this particle size distribution. In an embodiment, the separation unit 113 may set a length threshold for separating the particles of the carbide based on the range of the length of the particles of the carbide determined from the known particle size distribution of the particles of the carbide. Alternatively, in an embodiment, the separation unit 113 may detect the particles of the carbide from the image data binarized by the extraction unit 111 to create a particle size distribution. The separation unit 113 calculates the average value of the particles of the carbide determined from the created particle size distribution, and a length threshold for separating the particles of the carbide may be set based on the calculated average value. For example, the separation unit 113 may set a length twice the average value of the length of the particles of the carbide as a length threshold for separating the particles of the carbide.

As described for FIG. 2B, in the case where there are two portions having a length direction in each of the two intersecting directions, these portions are the particles of the carbide that differ from each other. In an embodiment, in the case where the separation unit 113 identifies that there is a first portion (for example, the portion A of FIG. 2B) in the shapes of carbides and a second portion (for example, the portion B of FIG. 2B) that intersects the first portion, the separation unit 113 can separate the first portion and the second portion as different particles of carbides.

### [Program for Detecting Carbide Particle]

In an embodiment of the present invention, it is possible to provide a program for performing the method for detecting the carbide particles described above. Alternatively, in an embodiment, the program can be provided as a stored recording medium. The program will be described with reference to FIG. 3 to FIG. 6.

The program causes the extraction unit 111 to read the SEM image of the steel material (S110). The program may cause the extraction unit 111 to read the SEM image of the steel material through the input device 120 and may cause the extraction unit 111 to read the SEM image stored in the storage device 140. Further, the program may cause the extraction unit 111 to read the SEM image stored in the server or the network drive via a network connected to the communication device 150.

The program causes the extraction unit 111 to binarize the image data of the read SEM image of the steel material to extract the shapes of the carbides (S130). Specifically, the program causes the extraction unit 111 to binarize the image data of the SEM image of the steel material for each pixel constituting the image data of the SEM image of the steel material based on the first luminance value (S131). More specifically, the program causes the extraction unit 111 to binarize the luminance value in the image data of the SEM image of the steel material, based on a predetermined value (hereinafter, also referred to as a first luminance value). More specifically, the program causes the extraction unit 111 to convert the luminance value of the portion having the luminance of the first luminance value or more (or larger than the first luminance value) in the image data of the SEM image to 1 (S133), and causes the extraction unit 111 to convert the luminance value of the portion having the luminance less than the first luminance value (or of the first luminance value or less) to 0 (S135). The program causes the extraction unit 111 to synthesize the part set in this way, and causes the extraction unit 111 to generate the image data obtained as a result of binarizing the image data of the SEM image of the steel material (S137). Consequently, the program can cause the extraction unit 111 to identify a portion having the luminance value 1 as a structure of a carbide (S139). In addition, the first luminance value may be a luminance value set in advance from the observation of the SEM image of the steel material, and may be a luminance value set by the user for the SEM image of the actual steel material to be processed by the extraction unit 111 and confirmed by the user on the display device 131. In an embodiment, the average value of the luminance of the pixel of interest and the luminance of the surrounding pixels may be set as the first luminance value (adaptive binarization).

The program causes the separation unit 113 to separate the particles of carbides from the image data binarized by the extraction unit 111 (S150). FIG. 5 is referred to. The program causes the separation unit 113 to generate a gradation toward the inside of the linear carbides (distribution of luminance) in accordance with a distance from a background having the luminance value 0 (S151). The program causes the separation unit 113 to set the highest luminance portion of the generated gradation as a center of the carbide (S153). The program causes the separation unit 113 to identify a first center of the first portion of the carbide and a second center of the second portion of the carbide as the center of the different particles of carbides, respectively (S155). Further, the program causes the separation unit 113 to identify the structure of the carbide including the first center and the structure of the carbide including the second center as different particles of carbides (S157).

Referring to FIG. 6, a method for separating the particles of the carbide from the binarized image data based on the shape of the carbides will be explained. The program causes the separation unit 113 to detect a contour of the structure of binarized carbide (S151A). The program causes the separation unit 113 to detect a position where the width direction is shortened as the boundary of the two particles of the carbides (separation point) in the binarized structure of the carbide (S153A). The program causes the separation unit 113 to identify the two particles of the carbides based on the boundary (S155A).

### Modified Example of Program for Detecting Carbide Particle

There is a particle size distribution in the particles of the carbide, and the particles of the carbide have a range of length from this particle size distribution. In an embodiment, the program may cause the separation unit 113 to set a length threshold to separate the particles of the carbide based on the range of the length of the particles of the carbide determined from the known particle size distribution of the particles of the carbide. Alternatively, in an embodiment, the program may cause the separation unit 113 to detect the particles of the carbide from the image data binarized by the extraction unit 111 to create a particle size distribution. The program may cause the separation unit 113 to calculate the average value of the particles of the carbide determined from the created particle size distribution, and to set a length threshold for separating the particles of the carbide based on the calculated average value. For example, the program may cause the separation unit 113 to set a length twice the average value of the length of the particles of the carbide as a length threshold for separating the particles of the carbide.

As described for FIG. 2B, in the case where there are two portions having a length direction in each of the two intersecting directions intersecting, these portions are the particles of the carbide that differ from each other. In an embodiment, the program may separate a first portion of a shape of the carbide (for example, the portion A of FIG. 2B) from a second portion (for example, the portion B of FIG. 2B) intersecting the first portion in the case where the separation unit 113 identifies that there is a first portion and a second portion intersecting the first portion, and the first portion and the second portion as the different particles of carbides.

### Second Embodiment: Apparatus for Detecting Carbide Particle

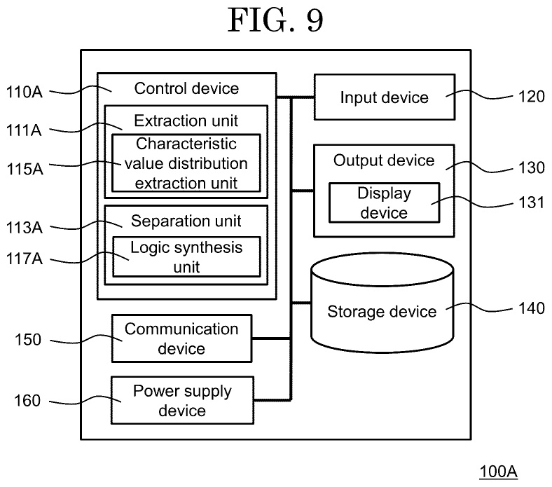

As a second embodiment of the present invention, an apparatus for detecting the carbide particle is described which separates particles of carbides in view of distribution of characteristics of the structure of the carbides when separating particles of carbides from binarized image data based on the watershed. FIG. 9 is a block diagram showing an apparatus 100A for detecting the carbide particle according to an embodiment of the present invention. The apparatus 100A for detecting the carbide particle includes, for example, a control device 110A, the input device 120, the output device 130, the storage device 140, the communication device 150, and the power supply 160. Further, in an embodiment, the apparatus 100A for detecting the carbide particle, for example, further comprises an extraction unit 111A and a separation unit 113A. In the present embodiment, the extraction unit 111A further includes a characteristic value distribution extraction unit 115A. The separation unit 113A further includes a logic synthesis unit 117A.

The control device 110A consists of a known central processor (CPU), an operating system (OS) and a control program or module for controlling the apparatus 100A for detecting the carbide particle. Alternatively, the control device 110A may be provided as one program including the OS and the control program or module. The control program or module constituting the control device 110A is stored in the storage device 140 and is executed by the CPU.

In FIG. 9, as an embodiment, although the control device 110A is configured to include the extraction unit 111A and the separation unit 113A, the extraction unit 111A and the separation unit 113A may be provided together with the control device 110A without being included in the control device 110A.

The extraction unit 111A is composed of a program or module for extracting the shapes of the carbides from image data of a SEM image of a steel material. Further, in the present embodiment, the extraction unit 111A further includes the characteristic value distribution extraction unit 115A. The characteristic value distribution extraction unit 115A is composed of a program or module for extracting the distribution of characteristic values of carbides from the image data of the SEM image of the steel material. Further, the extraction unit 111A includes a program or module for binarizing the image data of the SEM image of the steel material to extract the shapes of the carbides. In FIG. 9, although the characteristic value distribution extraction unit 115A is included in the extraction unit 111A, the characteristic value distribution extraction unit 115A may be a program or a module independent from the extraction unit 111A.

The program or module constituting the extraction unit 111A is stored in the storage device 140 and executed by the CPU. In an embodiment, the extraction unit 111A binarizes the luminance value in the image data of the SEM image of the steel material based on a predetermined value (hereinafter, also referred to as a first luminance value). More specifically, the luminance value of the portion having the luminance of the first luminance value or more (or larger than the first luminance value) in the image data of the SEM image is converted to 1, and the luminance value of the portion having the luminance less than the first luminance value (or of the first luminance value or less) is converted to 0. By such a process, the extraction unit 111A can identify the portion having the luminance value 1 as a structure of the carbide. In addition, the first luminance value may be a luminance value set in advance from observation of the SEM image of the steel material, or may be a luminance value set by the user for the actual SEM image of the steel material to be processed by the extraction unit 111A, and confirmed by the user on the display device 131. In an embodiment, the average value of the luminance of the pixel of interest and the luminance of the surrounding pixels may be set as the first luminance value (adaptive binarization).

The programs or modules constituting the characteristic value distribution extraction unit 115A are stored in the storage device 140 and executed by the CPU. In an embodiment, examples of the characteristic value of the carbides extracted by the characteristic value distribution extraction unit 115A include the luminance value of the carbide in the image data of the SEM image of the steel material, a depth of the carbide of the steel material, and element concentrations detected by an energy dispersive X-ray spectroscopy (EDS) or an electron probe microanalyzer (EPMA) or the like, but are not limited thereto. If the characteristic value of the carbide is the luminance value of the carbide in the image data of the SEM image of the steel material, the characteristic value distribution extraction unit 115A extracts the distribution of the luminance value of the carbide from the image data of the SEM image of the steel material, and stores the distribution in the storage device 140. Alternatively, if the characteristic value of the carbide is the depth of the carbide of the steel material, the characteristic value distribution extraction unit 115A extracts the distribution of the depth of the carbide from the data of the atomic force microscopy (AFM) of the steel material, and stores the distribution in the storage device 140. Alternatively, if the characteristic value of the carbide is the element concentration, the characteristic value distribution extraction unit 115A extracts the distribution of the element concentration of the carbide from the data of the EDS or the EPMA steel of the material, and stores the distribution in the storage device 140.

The separation unit 113A is composed of a program or a module for separating the particles of carbides from the binarized images by the extraction unit 111A. In this embodiment, the extraction unit 111A further includes a logic synthesis unit 117A. The program or module constituting the separation unit 113A is stored in the storage device 140 and executed by the CPU. In an embodiment, the separation unit 113A separates the structure of the carbide identified by the extraction unit 111A into the particles of the carbide. In an embodiment, the separation unit 113A separates the particles of the carbide from the binarized image data based on a shape of the watershed. In the case of separating the particles of carbides from the binarized image data based on the watershed, the separation unit 113A generates a gradation (distribution of luminance) toward the inside of the linear carbides in accordance with a distance from the background having the luminance value of 0.

In the present embodiment, the gradation (distribution of luminance) toward the inside of the carbide generated by the separation unit 113A and the distribution of characteristic values of the carbide extracted by the characteristic value distribution extraction unit 115A are synthesized by the logic synthesis unit 117A to generate a logic synthesized image. The characteristic value distribution extraction unit 115A is composed of a program or module for generating a logic synthesized image by synthesizing the gradation (distribution of luminance) toward the inside of the carbide and the distribution of characteristic values of the carbide. The characteristic value distribution extraction unit 115A synthesizes the gradation (distribution of luminance) toward the inside of the carbide and the distribution of the characteristic values of the carbide and generates a logical product image or a logic add image. In an embodiment, the characteristic value distribution extraction unit 115A may compare the logical product image with the logic add image and select one which has the larger difference between the maximum value and the minimum value of the luminance value in the logic synthesized image as the logic synthesized image used for the following process.

The separation unit 113A converts the luminance value of the portion of the generated logic synthesized image that is less than the threshold of the predetermined luminance to 0. Thus, the separation unit 113A extracts only the gradation part of the logic synthesized image that is more than or equal to the threshold of the predetermined luminance among the gradation part of the logic synthesized image. The separation unit 113A sets the portion with the highest luminance to the center of the carbides among the gradation parts of the extracted logic synthesized images. For example, in FIG. 2B, since the center is set in each of the portion A and the portion B of the carbide, the separation unit 113A can identify the center of the portion A of the carbide and the center of the portion B as the center of the particles of different carbides, respectively. Furthermore, the separation unit 113A can identify a structure of the carbide containing the center of the portion A and a structure of the carbide containing the center of the portion B as the different particles of carbides, respectively.

In the case of separating the particles of the carbide from the binarized image based on a shape of the carbides, the separation unit 113A detects a contour of the binarized structure of the carbide. Since the structure of the carbide has a linear structure, it has a length direction and a width direction. The separation unit 113A detects a position where the width direction is shortened as a boundary of the particles of the two carbides (separation point) in the structure of the binarized carbides, and can distinguish the two particles of the carbides.

Further, there is a particle size distribution in the particles of the carbide, and the particles of the carbide have a range of length from this particle size distribution. In an embodiment, the separation unit 113A may set a length threshold for separating the particles of the carbide based on the range of the length of the particles of the carbide determined from the known particle size distribution of the particles of the carbide.

Further, as described for FIG. 2B, in the case where there are two portions having a length direction in each of the two intersecting directions, these portions are the different particles from each other of carbides. In an embodiment, in the case where the separation unit 113A identifies that there are a first portion of the form of carbides (for example, the portion A of FIG. 2B) and a second portion intersecting the first portion (for example, the portion B of FIG. 2B), the first portion and the second portion can be separated as the different particles of carbides.

The input device 120, the output device 130, the storage device 140, the communication device 150, and the power supply device 160 may have the same configuration as that described in the apparatus 100 for detecting the carbide particle, and a detailed description thereof will be omitted.

### [Method for Detecting Carbide Particle]

A method for detecting carbide particles using the apparatus 100A for detecting the carbide particle according to the present invention described above will be described.

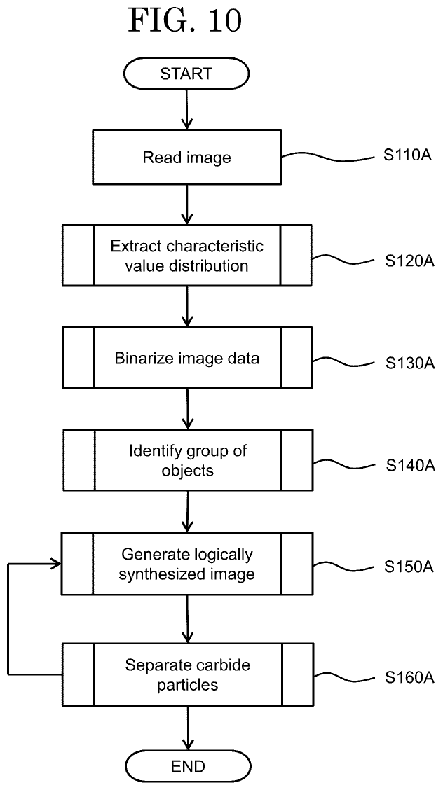

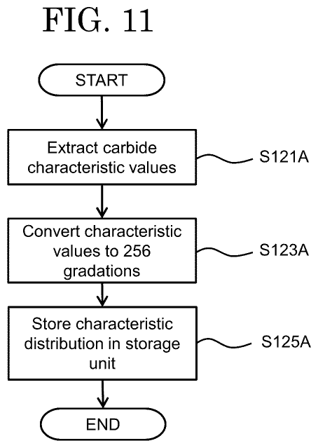

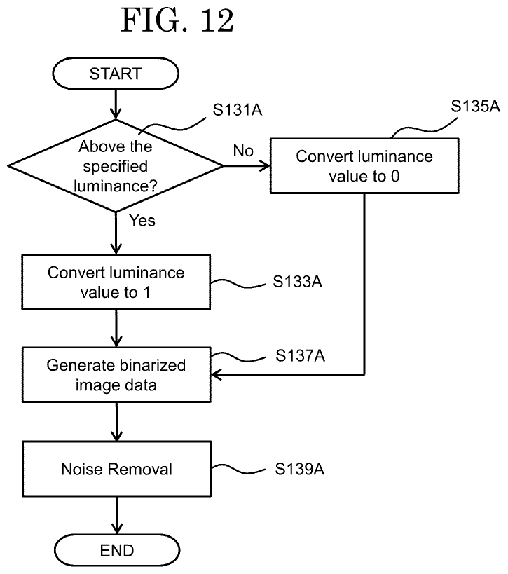

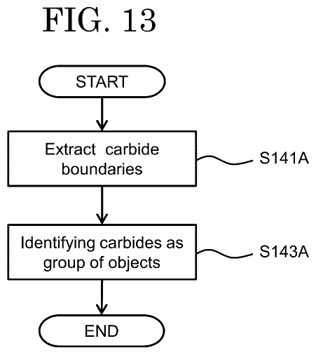

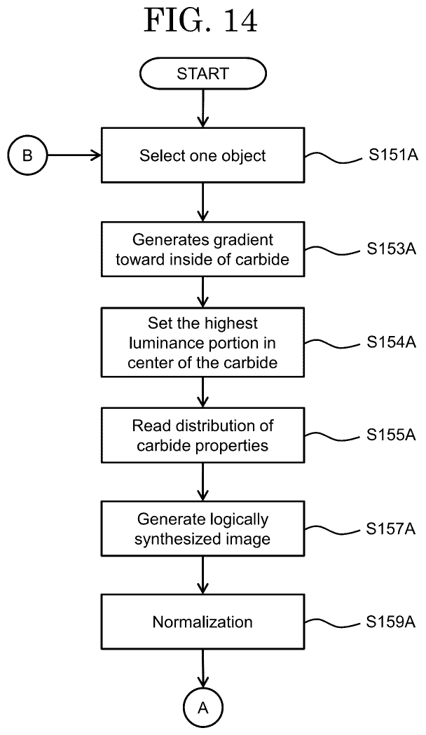

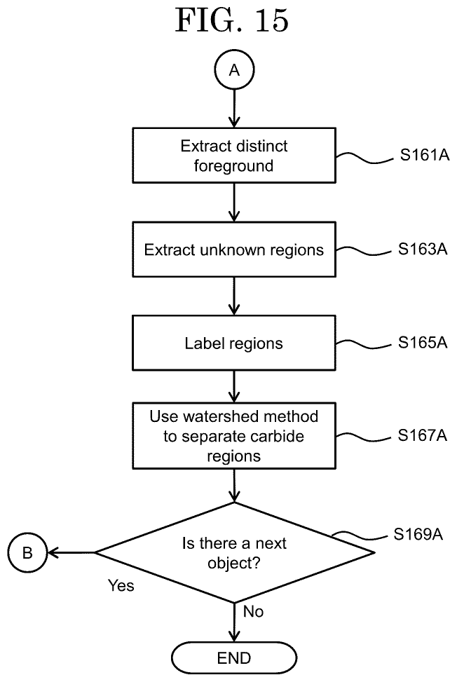

FIG. 10 to FIG. 15 are flow diagrams of a method for detecting carbide particles according to an embodiment of the present invention. The extraction unit 111A reads a SEM image of the steel material (S110A). The SEM image of the steel material may be read through the input device 120, and the extraction unit 111A may read the SEM image stored in the storage device 140. Further, the SEM image stored in the server or the network drive may be read by the extraction unit 111A through the network connected to the communication device 150.

The characteristic value distribution extraction unit 115A extracts the distribution of characteristic values of carbides from the image data of the SEM image of the steel material which the extraction unit 111A has read (S120A). Specifically, the characteristic value distribution extraction unit 115A extracts the characteristic values of the carbides from the image data of the SEM image of the steel material (S121A). For example, the characteristic value distribution extraction unit 115A extracts the luminance value of the carbides from the image data of the SEM image of the steel material. Alternatively, in an embodiment, the characteristic value distribution extraction unit 115A may read the data of an atomic force microscopy (AFM) of the steel material corresponding positionally with the SEM image of the steel material, and extract a depth of carbides from AFM data. Alternatively, in an embodiment, the characteristic value distribution extraction unit 115A reads the data of EDS or EPMA of the steel material positionally corresponding to the SEM image of the steel material, and may be extract an element concentration of carbides from the data of EDS or EPMA of the steel material.

The characteristic value distribution extraction unit 115A converts the extracted luminance value of the carbide, or the value of the depth of the carbide or the element concentration of the carbide to 256 gradations, a distribution data of the luminance value of the carbide, a distribution data of the depth of the carbide, or a distribution data of the element concentration of the carbide positionally corresponding to the SEM image of the steel material (S123A). Further, the characteristic value distribution extraction unit 115A stores the distribution data of the luminance value of the carbide, the distribution data of the depth of the carbide, or the element concentration of the carbide positionally corresponding to the generated SEM image of the steel material in the storage device 140 (S125A).

The extraction unit 111A binarizes the image data of the SEM image of the read steel material to extract the shapes of the carbides (S130A). Specifically, the extraction unit 111A binarizes the image data of the SEM image of the steel material for each pixel constituting the image data of the SEM image of the steel material, based on the first luminance value (S131A). More specifically, the extraction unit 111A binarizes the luminance value in the image data of the SEM image of the steel material, with a predetermined value (hereinafter, also referred to as the first luminance value) as a reference. More specifically, the luminance value of the portion having the luminance of the first luminance value or more (or larger than the first luminance value) in the image data of the SEM image is converted to 1 (S133A), and the luminance value of the portion having the luminance less than the first luminance value (or of the first luminance value or less) is converted to 0 (S135A). Synthesizing the part set in this way, the extraction unit 111A generates image data obtained by binarizing image data of the SEM image of the steel material (S137A).

In order to extract boundaries of carbides in the binarized image data, the extraction unit 111A removes noise from the binarized image data (S139A). For example, using a morphological gradient, or smoothing filtering (averaging filter, Gaussian filter, or median filter, or the like), the extraction unit 111A may remove noise from binarized image data of the SEM image of the steel material.

The extraction unit 111A identifies a group of objects in the binarized image data in which noise is removed (S140A). The extraction unit 111A identifies a region where the luminance value is 0 as a distinct background in the binarized image data from which the noise is removed to extract the boundaries of carbides (S141A). As a consequence of extracting the boundaries of the carbides by the extraction unit 111A, the portion with the luminance value 1 can be identified as the group of the object of carbides (S143A). The first luminance value may be a luminance value set in advance from the observation of the SEM image of the steel material, or may be a luminance value set by the user for the actual SEM image of the steel material to be processed by the extraction unit 111A, and confirmed by the user on the display device 131. In an embodiment, the average value of the luminance of the pixel of interest and the luminance of the surrounding pixels may be set as the first luminance value (adaptive binarization).

The separation unit 113A generates logic synthesized images for the group of the object identified by the extraction unit 111A (S150A). Specifically, the separation unit 113A selects one object from the group of the object (S151A), and generates a gradient toward the inside of the linear carbide (distribution of luminance) in accordance with a distance from the background having a luminance value 0, and is used to perform distance transformation of the carbide included in the selected one object (S153A). The separation unit 113A sets the portion with the highest luminance of the generated gradient as the center of the carbide to obtain the distance transformed image data (S154A).

The separation unit 113A reads the distribution of the characteristic value of the carbides stored in the storage device 140 (S155A). The separation unit 113A generates a logic synthesized image by logically synthesizing the distance transformed image data and the read distribution of characteristic values of the carbides (S157A). In this embodiment, the logical product or the logical add can be used as the logic synthesis. The separation unit 113A may generate a logic synthesized image by calculating the logical product of the distance transformed image data and the distribution of the characteristic values of the carbide, and may generate a logic synthesized image by calculating a logical add of the distance transformed image data and the distribution of the characteristic values of the carbide. Alternatively, the separation unit 113A may calculate the logical product and the logical add to output a logic synthesized image having a larger effect on the separation of the carbides. In an embodiment, the Hadamard product may be used as a logical product. In addition, in an embodiment, a weighted sum may be used as a logical add. The separation unit 113A normalizes the generated logic synthesized images (S159A).

The separation unit 113A separates the particles of carbides based on the normalized logic synthesized images (S160A). Specifically, the separation unit 113A extracts distinct foreground regions for the normalized logic synthesized images (S161A). In an embodiment, a threshold of 0.6 to 0.8 can be set as a threshold for extracting distinct foreground regions. In addition, the separation unit 113A extracts unknown regions for the normalized logic synthesized images (S163A). Consequently, the separation unit 113A determines a region of a reliable foreground and labels each of the determined regions (S165A). The separation unit 113A applies a watershed algorithm to divide the structure of a carbide containing the first center and the structure of carbide containing the second center, for the labeled regions, and distinguish them as different particles of carbides (S167A). The separation unit 113A performs S151A to S167A processes for all objects included in the group of the object, and separates and distinguishes the particles of the carbides for all objects (S169A).

### [Program for Detecting Carbide Particle]

In an embodiment of the present invention, it is possible to provide a program for performing the method for detecting the carbide particles described above. Alternatively, in an embodiment, the program can be provided as a stored recording medium. The program will be described with reference to FIG. 10 to FIG. 15.

The program causes the extraction unit 111A to read the SEM image of the steel material(S110). The program may cause the extraction unit 111A to read the SEM image of the steel material through the input device 120 and may cause the extraction unit 111A to read the SEM image stored in the storage device 140. The program may cause the extraction unit 111A to read the SEM image stored in the server or the network drive through a network connected to the communication device 150.

The program causes the extraction unit 111A to extract the distribution of the characteristic values of carbides from the read image data of the SEM image of the steel material (S120A). Specifically, the program causes the characteristic value distribution extraction unit 115A to extract the characteristic values of carbides from the image data of the SEM image of the steel material (S121A). For example, the program causes the characteristic value distribution extraction unit 115A to extract the luminance value of the carbides from the image data of the SEM image of the steel material. Alternatively, the program causes the characteristic value distribution extraction unit 115A to extract the luminance value of the carbides from the image data of the SEM image of the steel material. In an embodiment, the program causes the characteristic value distribution extraction unit 115A to read the data of the atomic force microscopy (AFM) of the steel material positionally corresponding to the SEM image of the steel material, and extract a depth of the carbides from the AFM data. Alternatively, in an embodiment, the program causes the characteristic value distribution extraction unit 115A to read data of EDS or EPMA of the steel material corresponding to the SEM image of the steel material, and extract an element concentration of carbides from the data of the EDS or the EPMA.

The program causes the characteristic value distribution extraction unit 115A to convert the extracted luminance value of the carbide, the value of the depth of the carbide, or the element concentration of the carbide into 256 gradations, and generate the distribution data of the luminance value of the carbide, or distribution data of depths of carbides or element concentrations of carbides positionally corresponding to the SEM image of the steel material (S123A). Further, the program causes the characteristic value distribution extraction unit 115A to store the distribution data of the luminance value of the carbide, or the distribution data of the depth of the carbide or the element concentration of the carbide corresponding to the generated SEM image of the steel material in the storage device 140 (S125A).

The program causes the extraction unit 111A to binarize the read image data of the SEM image of the steel material to extract shapes of carbides (S130A). Specifically, the program causes the extraction unit 111A to binarize the image data of the SEM image of the steel material for each pixel constituting the image data of the SEM image of the steel material, based on the first luminance value (S131A). More specifically, the program causes the extraction unit 111A to binarize the luminance value in the image data of the SEM image of the steel material, with a predetermined value (hereinafter, also referred to as the first luminance value) as a reference. More specifically, the program causes the extraction unit 111A to convert the luminance value of the portion having the luminance of the first luminance value or more (or larger than the first luminance value) in the image data of the SEM image to 1 (S133A), and causes the extraction unit 111A to convert the luminance value of the portion having the luminance less than the first luminance value (or of the first luminance value or less) to 0 (S135A). The program synthesizes the part set in this way, and causes the extraction unit 111A to generate image data obtained by binarizing the image data of the SEM image of the steel material (S137A).

In order to extract the boundaries of carbides in the binarized image data, the program causes the extraction unit 111A to remove noise from the binarized image data (S139A). For example, the program may use a morphological gradient or smoothing filtering (averaging filter, Gaussian filter, or median filter) to cause the extraction unit 111A to remove noise from the image data obtained by binarizing the image data of the SEM image of the steel material.

The program causes the extraction unit 111A to identify the group of the object in the binarized image data from which noise is removed (S140A). The program causes the extraction unit 111A to identify the region where the luminance value is 0 as a distinct background in the binarized image data in which the noise is removed to extract the boundaries of carbides (S141A). The program causes the extraction unit 111A to extract the carbide boundaries, and as a result, the portions having the luminance value 1 can be identified as the group of the object of the carbides (S143A). In addition, the first luminance value may be a luminance value set in advance based on the observation of the SEM image of the steel material, or may be a luminance value set by the user for the actual SEM image of the steel material that is processed by the extraction unit 111A and that the user has confirmed on the display device 131. In an embodiment, the program may set the average value of the luminance of the pixel of interest and the luminance of the surrounding pixels as the first luminance value (adaptive binarization).

The program causes the separation unit 113A to generate logic synthesized images for the group of the object identified by the extraction unit 111A (S150A). Specifically, the program causes the separation unit 113A to select one object from the group of the object (S151A), to generate a gradation toward the interior of linear carbides (distribution of luminance) in accordance with a distance from the background having the luminance value 0, and to perform distance transformation on the carbides included in one selected object (S153A). The program causes the separation unit 113A to set the highest luminance part of the generated gradation as a center of the carbide, and to acquire the distance transformed image data (S154A).

The program causes the separation unit 113A to read the distribution of characteristic values of the carbides stored in the storage device 140 (S155A). The program causes the separation unit 113A to logically synthesize the distance transformed image data and the read distribution of the characteristic values of the carbide to generate a logic synthesized image (S157A). In this embodiment, the logical product or the logical add can be used as the logical synthesis. The program may cause the separation unit 113A to obtain a logical product of the distance transformed image data and the distribution of the characteristic values of the carbide to generate a logic synthesized image, or may cause the separation unit 113A to obtain a logical add of the distance transformed image data and the distribution of the characteristic values of the carbide to generate a logic synthesized image. Alternatively, the program may cause the separation unit 113A to calculate a logical product and a logical add, and output the logic synthesized image that has the greater effect on separating carbides. In an embodiment, a Hadamard product may be used as a logical product. In addition, in an embodiment, a weighted sum may be used as a logical add. The program causes the separation unit 113A to normalize the generated logic synthesized images (S159A).

The program causes the separation unit 113A to separate the particles of the carbide based on the normalized logic synthesized images (S160A). Specifically, the program causes the separation unit 113A to extract distinct foreground regions for the normalized logic synthesized images (S161A). In an embodiment, a threshold of 0.6 to 0.8 can be set as a threshold for extracting distinct foreground regions. In addition, the program causes the separation unit 113A to extract unknown regions for the normalized logic synthesized images (S163A). Consequently, the program causes the separation unit 113A to determine a certain foreground region and assign a label to each determined region (S165A). The program causes the separation unit 113A to apply a watershed algorithm to separate the labeled region into a structure of the carbide including a first center and a structure of the carbide including a second center, and to identify each of them as the different particles of the carbide (S167A).

The program causes the separation unit 113A to perform S151A to S167A process for all objects included in the group of the object, and separate and identify the particles of carbides for all objects (S169A).

### EXAMPLE

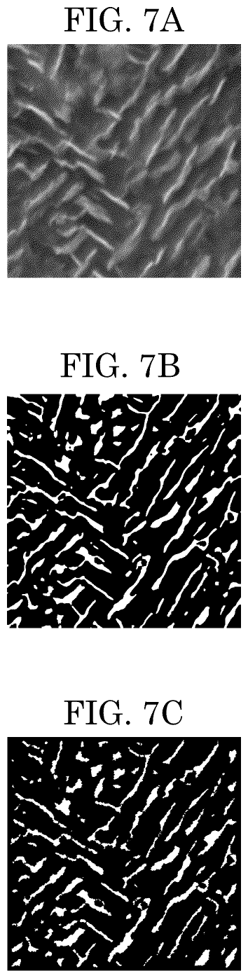

An example of detecting carbide particles using the apparatus 100 for detecting carbide particles of the first embodiment described above will be shown below. FIG. 7A is a SEM image of a surface of the steel structure exposed by etching. The extraction unit 111 reads the SEM image of FIG. 7A, and binarizes the image data of the SEM image of the steel material to extract shapes of the carbides. FIG. 7B is binarized image data. The separation unit 113 separated the structures of carbides and identified them as particles of carbides as shown in FIG. 7C.

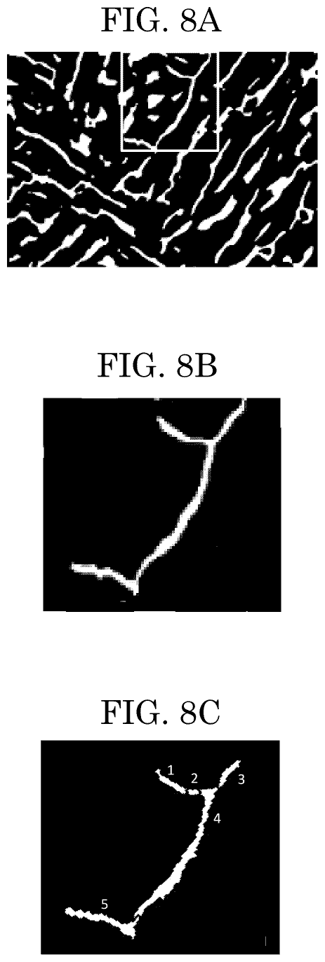

FIG. 8A to FIG. 8C are referred to. FIG. 8A is a binarized view of the SEM image of a steel material. FIG. 8B is an enlarged view of a part enclosed by a square in FIG. 8A. For structures displayed as one carbide in FIG. 8B, as a result of identifying the particles of the carbide by the separation unit 113, the particles of five carbides were identified as shown in FIG. 8C.

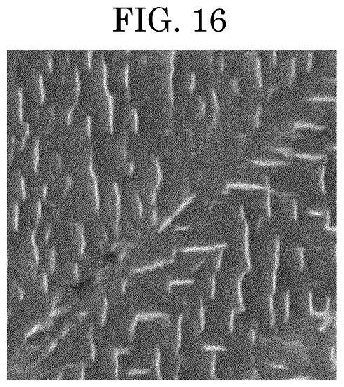

An example of detecting carbide particles using the apparatus 100A for detecting the carbide particle of the second embodiment described above will be described below. FIG. 16 is a SEM image of a surface of a steel structure exposed by etching. The characteristic value distribution extraction unit 115A reads the SEM image of FIG. 16, extracts the luminance value of the carbides from the image data of the SEM image of the steel material, converts the extracted luminance value of the carbides to 256 gradations, and generates distribution data of the luminance value of the carbides positionally corresponding to the SEM image of the steel material.

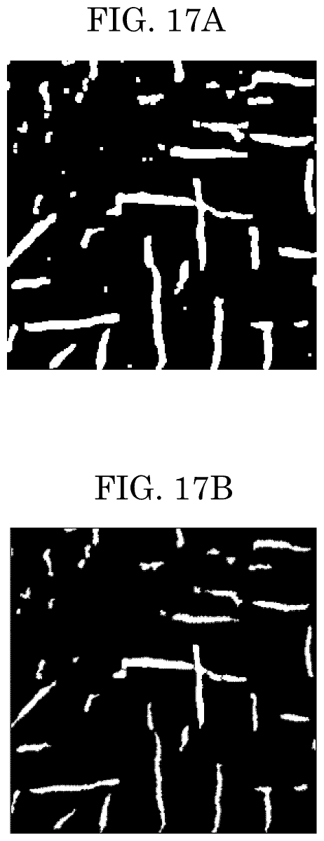

Further, the extraction unit 111A reads the SEM image of FIG. 16, binarized the image data of the SEM image of the steel material, and extracted the shape of the carbides. FIG. 17A is binarized image data. The separation unit 113A identified the group of the object of the carbides as shown in FIG. 17B.

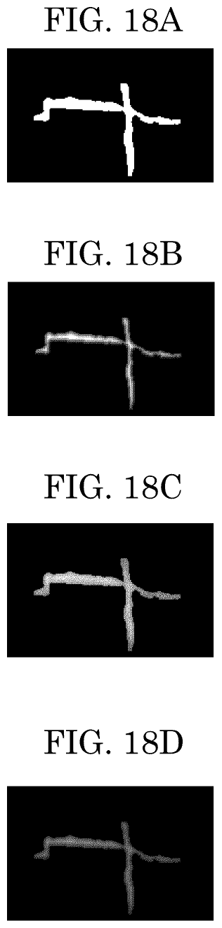

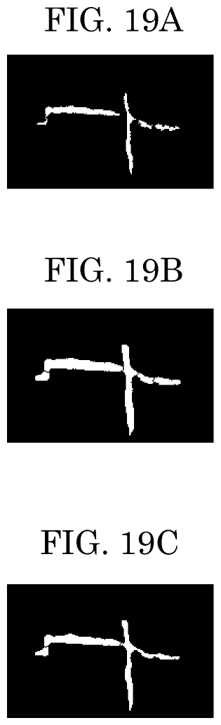

FIG. 18A to FIG. 19C are referred to. FIG. 18A is a diagram showing an object of one carbide in which image data of the SEM image of the steel material was binarized. FIG. 18B shows a diagram in which the separation unit 113A performs the distance transformation, and FIG. 18C is a SEM image of the steel material of one carbide extracted from FIG. 16 corresponding to one carbide of FIG. 18A. Further, FIG. 18D is a logic synthesized image generated by the logical product of the distribution of the characteristic values of the carbide and the image data which was distance transformed by the separation unit 113A.

FIG. 19A is a diagram in which a distinct foreground area is extracted for logic synthesized images normalized by the separation unit 113A. FIG. 19B is a diagram in which the separation unit 113A applied the watershed algorithm to divide the carbide structures and identify each as the different particles of the carbide. On the other hand, FIG. 19C is a diagram showing the result of applying the watershed algorithm using only the data that has been distance transformed by the separation unit 113A. Comparing to FIG. 19B and FIG. 19C, it has been shown that the accuracy of dividing the particles of the carbide is further improved by using the method for detecting the carbide particle of the second embodiment.

The case of using the SEM image of the steel material by a scanning electron microscope in the above embodiments and examples has been described. Even if microscopes other than a scanning electron microscope are used, it is also possible to use a microscope such as a transmission electron microscope (TEM), a scanning transmission electron microscope (STEM), and an atomic force microscope (AFM) which are capable of observing the crystal grains of the steel material.

Although an embodiment of the present invention has been described above with reference to the drawings, the present invention is not limited to the above embodiment and can be modified as appropriate without departing from the spirit of the present invention. For example, based on the apparatus for detecting the carbide particle of the steel material, the program for detecting the carbide particle of the steel material, and the method for detecting the carbide particle of the steel material of the present embodiment, any addition, deletion or design modification of components by a person skilled in the art is included within the scope of the present invention as long as it maintains the gist of the present invention. Furthermore, the embodiments described above can be appropriately combined as long as they do not contradict each other, and the technical matters common to each embodiment are included in each embodiment without explicit description.

It is to be understood that other operational effects different from those provided by the aspects of each of the embodiments described above are naturally provided by the present invention as to those apparent from the description of the present specification or those which can be easily predicted by persons skilled in the art.

An embodiment of the present invention provides a properly distinguishable apparatus for detecting carbide particles in the steel material. Alternatively, an embodiment of the present invention provides a method for detecting carbide particles that can properly discriminate carbide particles in the steel material. Alternatively, an embodiment of the present invention provides a program for detecting carbide particles that can properly discriminate carbide particles in the steel material.

What is claimed is:

1. An apparatus for detecting a carbide particle comprising:
an extraction unit for binarizing image data of a microscope image of a steel material to extract shapes of carbides; and
a separation unit for separating particles of the carbides from the binarized image data based on watersheds or the shapes of the carbides.

2. The apparatus for detecting the carbide particle according to claim 1, wherein
the separation unit sets a length threshold for separating the particles of the carbide based on a length range of the particles of the carbide determined from a known particle size distribution of the particles of the carbides.

3. The apparatus for detecting the carbide particle according to claim 1, wherein
the separation unit separates a first portion and a second portion as different particles of the carbides when the separation unit identifies that the first portion of the shapes of the carbides and the second portion intersecting the first portion exist.

4. The apparatus for detecting the carbide particle according to claim 1, wherein
the extraction unit includes a characteristic value distribution extraction unit,
the characteristic value distribution extraction unit extracts a distribution of characteristic values of the carbides from the image data of the microscope image of the steel material,
the separation unit executes a logic synthesis of the binarized image data and the distribution of characteristic values of the carbides to generate a logic synthesized image, and
the separation unit separates the particles of the carbides by using the logic synthesized image based on the watersheds.

5. A method for detecting a carbide particle comprising:
using an apparatus for detecting the carbide particle including an extraction unit and a separation unit,
wherein the extraction unit binarizes image data of a microscope image of a steel material to extract shapes of carbides, and
the separation unit separates particles of the carbides from the binarized image data based on watersheds or the shapes of the carbides.

6. The method for detecting the carbide particle according to claim 5, wherein
the separation unit sets a length threshold for separating particles of the carbide based on a length range of the particles of the carbide determined from a known particle size distribution of the particles of the carbides.

7. The method for detecting the carbide particle according to claim 5, wherein
the separation unit separates a first portion and a second portion as different particles of the carbides when the separation unit identifies that the first portion of the shapes of the carbides and the second portion intersecting the first portion exist.

8. The method for detecting the carbide particle according to claim 5, wherein
the extraction unit includes a characteristic value distribution extraction unit,
the characteristic value distribution extraction unit extracts a distribution of characteristic values of the carbides from the image data of the microscope image of the steel material,
the separation unit executes a logic synthesis of the binarized image data and the distribution of characteristic values of the carbides to generate a logic synthesized image, and
the separation unit separates particles of the carbides by using the logic synthesized image based on the watersheds.

9. A storage media storing a program for detecting a carbide particle comprising:
causing a computer to binarize image data of a microscope image of a steel material to extract shapes of carbides; and
causing the computer to separate particles of the carbides from the binarized image data based on watersheds or the shapes of the carbides.

10. The storage media storing the program for detecting the carbide particle according to claim 9, wherein
the program causes the computer to set a length threshold for separating particles of the carbide based on a length range of the particles of the carbide determined from a known particle size distribution of the particles of the carbides.

11. The storage media storing the program for detecting the carbide particle according to claim 9, wherein
the program causes the computer to separate a first portion and a second portion as different particles of the carbides when the computer identifies that the first portion of the shapes of the carbides and the second portion intersecting the first portion exist.

12. The storage media storing the program for detecting the carbide particle according to claim 9, wherein
the program causes the computer to extract a distribution of characteristic values of the carbides from the image data of the microscope image of the steel material,
the program causes the computer to execute a logic synthesis of the binarized image data and the distribution of characteristic values of the carbides to generate a logic synthesized image, and
the program causes the computer to separate particles of the carbides by using the logic synthesized image based on the watersheds.
